(19) United States
(12) Patent Application Publication
(10) Pub. No.: US 2023/0329152 A1
(43) Pub. Date: Oct. 19, 2023

(54) PLANT PROPAGATION

(71) Applicant: SYNGENTA CROP PROTECTION AG, Basel (CH)

(72) Inventor: Donald Penn Snow, Jacksonville, OR (US)

(73) Assignee: SYNGENTA CROP PROTECTION AG, Basel (CH)

(21) Appl. No.: 18/026,939

(22) PCT Filed: Sep. 16, 2021
(86) PCT No.: PCT/US2021/050579

§ 371 Date: Mar. 17, 2023

Related U.S. Application Data
(60) Provisional application No. 63/079,491, filed on Sep. 17, 2020.

(51) Int. Cl. A01G 2/10; A01H 6/38; A01H 4/00; A01G 22/60

(52) U.S. Cl. CPC A01G 2/10; A01H 6/385; A01H 4/008; A01G 22/60

(57) ABSTRACT

Methods of propagating and growing poinsettias in pots have allowed poinsettias to become the highest selling potted flowering plant. The present application relates generally to the field of plant propagation. In particular, a method of producing a pre-pinched, callused poinsettia cutting is provided. Also provided is a method to produce a poinsettia with improved growth habit. The invention also provides a pre-pinched, callused poinsettia cutting.

### RELATED APPLICATIONS

This application claims priority from provisional application 63/079,491 filed Sep. 17, 2020 and incorporated by reference in its entirety herein.

### FIELD OF THE INVENTION

The invention relates to compositions and methods for propagation of plants, such as Poinsettias. In particular, the invention relates to methods of pre-pinching callused cuttings.

### BACKGROUND

In nature, the poinsettia grows as a small tree up to twelve feet tall. Methods of propagating and growing poinsettias in pots have allowed poinsettias to become the highest selling potted flowering plant. Poinsettias are propagated by cuttings. Growers of Poinsettia (Euphorbia pulcherrima) purchase vegetative cuttings from suppliers in two different product forms: unrooted cutting (URC) or callused cutting (CC). Unrooted cuttings are harvested from stock plants of poinsettia according to size specifications and then are packaged and shipped to customers. Upon receipt of URCs, growers stick the cutting in media for rooting. Callused cuttings are harvested like URCs and then the supplier inserts the cutting into a media to initiate callusing (i.e., the first stage of adventitious rooting). After callus formation, the cuttings are removed from the media, washed, packaged, and shipped to customers. Upon receipt of CCs, growers stick the cutting in media and rooting speed is faster than with URCs. Following rooting, plants are pinched by the grower to promote lateral branching. Pinching is labor intensive, messy due to poinsettia latex sap, and can spread disease. Described is a new product form which includes a poinsettia cutting that is callused and pre-pinched by the supplier before shipping to customer. The benefits to the grower include the ability to direct stick the cutting into the final container, no pinching required, and faster finish time.

Poinsettia cuttings are normally rooted and planted in a pot allowing the plant to establish roots before the plant is pinched to promote branching. The Eche Poinsettia Manual by Paul Ecke III et al. published by Ball Publishing (herein incorporated by reference) teaches that “Before pinching poinsettias, it is advisable to have adequate root systems established. The size of growing container and the volume of medium influence the time required for rooted cuttings to develop adequate roots prior to pinching.” Provided herein are methods to grow plants with improved growth habit by pinching unrooted cuttings.

### SUMMARY

In some embodiments, a method of growing a poinsettia with improved growth habit from a callused, pinched cutting is provided comprising the stems of cutting a stem tip cutting, callusing the cutting, pinching the stem tip before planting and planting the cutting in the final container, wherein the poinsettia produced by callusing and pinching before planting has an improved growth habit.

In some embodiments, a method of growing a poinsettia with improved growth habit from a callused, pinched cutting is provided comprising the stems of cutting a stem tip cutting, callusing the cutting, pinching the stem tip before planting, planting the cutting in the final container, wherein the poinsettia produced by callusing and pinching before planting has an improved growth habit, wherein the improved growth habit is 4 cm taller stems than a poinsettia pinched after planting and rooting.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit has about 3 fewer branches than a poinsettia pinched after planting and rooting.

In some embodiments, a method of growing a poinsettia with improved growth habit from a callused, pinched cutting is provided comprising the stems of cutting a stem tip cutting, callusing the cutting, pinching the stem tip before planting and planting the cutting in the final container, wherein the poinsettia produced by callusing and pinching before planting has an improved growth habit, wherein the improved growth habit is 14 days faster to retail size than a poinsettia pinched after planting and rooting.

In some embodiments, a poinsettia plant with improved growth habit is provided produced by a method of growing from a callused, pinched cutting comprising the stems of cutting a stem tip cutting, callusing the cutting, pinching the stem tip before planting and planting the cutting in the final container.

In some embodiments, a method of producing a callused, pre-pinched Poinsettia cutting is provided comprising the stems of cutting a stem tip cutting and callusing the cutting and pinching the stem tip before planting.

In some embodiments, a callused, pre-pinched Poinsettia cutting is provided produced by a method comprising the stems of cutting a stem tip cutting, callusing the stem tip cutting, and pinching the stem tip before planting.

In some embodiments, a callused, pre-pinched Poinsettia cutting is provided.

### DETAILED DESCRIPTION

Pinching refers to cutting a plant stem. Pinching can be performed with fingers, knife, scissors, pruning shears, or other sharp objects. Pinching results in a more branched plant habit. Where one stems is pinched, two new stems grow from the next stem node. Pinching also produces compact growth by reducing plant height. Crop time is the time from sticking the Poinsettia cutting in the final container to the opening of the cyathia.

Callus is formed by undifferentiated cells at a wound site often initiated under high moisture conditions. Callus formation protects the cutting from dehydration. Callus formation is a precursor to adventitious rooting. A standard Poinsettia cutting is 4.0 to 5.0 cm long when the cutting is harvested from the stock plant and has approximately four leaf nodes on the stem. Length is measured from the base of the cut stem to the shoot apical meristem. To create a pre-pinched callused Poinsettia cutting, the first step is to harvest a cutting from the stock plant that is 2.0 to 2.5 cm longer than a standard cutting (total length 6.0 to 7.0 cm) with one to three additional leaf nodes for a total of five to seven nodes. After harvest, the next step is callusing the cutting. The cutting of appropriate length and node count is stuck into a pre-made dibbled hole about 1 cm in diameter in an appropriate pre-wetted rooting medium (such as disinfected sand, rockwool, or peat moss). Rooting hormone [indole-3-butyric acid (IBA) or naphthaleneacetic acid (NAA)] may be applied as a liquid or powder dip to the base of the stem before sticking to accelerate the rooting process and improve uniformity. The rooting medium is usually contained in planting trays consisting of individual cells and these trays are on a raised bench in a greenhouse.

After sticking the cutting in the rooting medium, the cutting is misted with water regularly to retain turgidity. The ideal misting frequency should continually provide a film of water on the leaves and maintain humidity around 100%. Shade is applied over the propagation area to reduce stress on the cuttings and maintain 1,000-2,000 foot candles (200-300 μmol·m⁻²·s⁻¹) during the brightest part of the day. The 24-hour average daily temperature of the rooting medium should be 22-26° C. (72-78° F.). Optional fertilizers, fungicides, insecticides, and plant growth regulators can be applied.

Callus formation at the base of the cut stem begins about 7 to 10 days after sticking in the medium and root tips are visible 10 to 14 days after sticking, depending on media, air temperature, and medium temperature. Approximately 17 to 21 days after sticking, the cuttings have developed roots approximately 2 to 3 cm long, and are ready to be harvested, pinched, and packaged for shipping to the customer. In this process, a greenhouse worker removes the rooted callused cutting from the propagation medium and then rubs off any roots with their fingers, leaving only the callus at the base of the cutting. The next step is pinching the distal shoot apical meristem off the cutting, removing one to two leaf nodes and leaving a minimum of four leaf nodes on the cutting. The shoot-tip pinch can be done manually by hand or with a disinfected knife. The removed shoot-tip is discarded and the remaining basal portion of the cutting with a minimum of four leaf nodes is the finished product.

Plant height and branch number were compared between plants grown from pre-pinched, callused cutting and non-pre-pinched callused cuttings as descried in example 2.

TABLE 1
Branch Number and Plant Height

| | Week | Variety | | | Number of | Height |
|---|---|---|---|---|---|---|
| Expt. | Stuck | Name | Treatment | Pl. # | Branches | (cm) |
| Rep 1 | 30 | Mirage ™ | Non-pre- | 1 | 7 | 26 |
| | | Red | pinched | | | |
| Rep 1 | 30 | Mirage ™ | Non-pre- | 2 | 8 | 26 |
| | | Red | pinched | | | |
| Rep 1 | 30 | Mirage ™ | Non-pre- | 3 | 7 | 24 |
| | | Red | pinched | | | |
| Rep 1 | 30 | Mirage ™ | Non-pre- | 4 | 10 | 29 |
| | | Red | pinched | | | |
| Rep 1 | 30 | Mirage ™ | Non-pre- | 5 | 8 | 27 |
| | | Red | pinched | | | |
| Rep 1 | 30 | Mirage ™ | Pre-Pinched | 1 | 6 | 28 |
| | | Red | | | | |
| Rep 1 | 30 | Mirage ™ | Pre-Pinched | 2 | 4 | 31 |
| | | Red | | | | |
| Rep 1 | 30 | Mirage ™ | Pre-Pinched | 3 | 6 | 29 |
| | | Red | | | | |
| Rep 1 | 30 | Mirage ™ | Pre-Pinched | 4 | 6 | 29 |
| | | Red | | | | |
| Rep 1 | 30 | Mirage ™ | Pre-Pinched | 5 | 6 | 27 |
| | | Red | | | | |
| Rep 2 | 32 | Mirage ™ | Non-pre- | 1 | 8 | 23 |
| | | Red | pinched | | | |
| Rep 2 | 32 | Mirage ™ | Non-pre- | 2 | 7 | 27 |
| | | Red | pinched | | | |
| Rep 2 | 32 | Mirage ™ | Non-pre- | 3 | 10 | 19 |
| | | Red | pinched | | | |
| Rep 2 | 32 | Mirage ™ | Non-pre- | 4 | 7 | 22 |
| | | Red | pinched | | | |
| Rep 2 | 32 | Mirage ™ | Non-pre- | 5 | 8 | 21 |
| | | Red | pinched | | | |
| Rep 2 | 32 | Mirage ™ | Pre-Pinched | 1 | 2 | 25 |
| | | Red | | | | |
| Rep 2 | 32 | Mirage ™ | Pre-Pinched | 2 | 5 | 27 |
| | | Red | | | | |
| Rep 2 | 32 | Mirage ™ | Pre-Pinched | 3 | 5 | 29 |
| | | Red | | | | |
| Rep 2 | 32 | Mirage ™ | Pre-Pinched | 4 | 7 | 31 |
| | | Red | | | | |
| Rep 2 | 32 | Mirage ™ | Pre-Pinched | 5 | 6 | 27 |
| | | Red | | | | |

The time taken to produce a finished plant was compared between plants started from pre-pinched, callused cuttings and plants started from non-pre-pinched, callused cuttings as described in example 2.

TABLE 2
Time to finished plant.

| | | | | | | | | Days: | Days: | Days: |
|---|---|---|---|---|---|---|---|---|---|---|
| | | | | | | | | Stick | Pinch | Stick |
| | Week | Variety | | Pl. | Finish | Stick | Pinch | to | to | to |
| Expt. | Stuck | Name | Treatment | # | Date | date | date | Finish | Finish | Pinch |
| Rep | 30 | Mirage ™ | Non-pre- | 1 | Nov. 19, 2019 | Jul. 24, 2019 | Aug. 29, 2019 | 118 | 82 | 36 |
| 1 | | Red | pinched | | | | | | | |
| Rep | 30 | Mirage ™ | Non-pre- | 2 | Nov. 25, 2019 | Jul. 24, 2019 | Aug. 29, 2019 | 124 | 88 | 36 |
| 1 | | Red | pinched | | | | | | | |
| Rep | 30 | Mirage ™ | Non-pre- | 3 | Nov. 21, 2019 | Jul. 24, 2019 | Aug. 29, 2019 | 120 | 84 | 36 |
| 1 | | Red | pinched | | | | | | | |
| Rep | 30 | Mirage ™ | Non-pre- | 4 | Nov. 19, 2019 | Jul. 24, 2019 | Aug. 29, 2019 | 118 | 82 | 36 |
| 1 | | Red | pinched | | | | | | | |
| Rep | 30 | Mirage ™ | Non-pre- | 5 | Nov. 25, 2019 | Jul. 24, 2019 | Aug. 29, 2019 | 124 | 88 | 36 |
| 1 | | Red | pinched | | | | | | | |
| Rep | 30 | Mirage ™ | Non-pre- | 6 | Nov. 21, 2019 | Jul. 24, 2019 | Aug. 29, 2019 | 120 | 84 | 36 |
| 1 | | Red | pinched | | | | | | | |
| Rep | 30 | Mirage ™ | Non-pre- | 8 | Nov. 25, 2019 | Jul. 24, 2019 | Aug. 29, 2019 | 124 | 88 | 36 |
| 1 | | Red | pinched | | | | | | | |
| Rep | 30 | Mirage ™ | Non-pre- | 9 | Nov. 19, 2019 | Jul. 24, 2019 | Aug. 29, 2019 | 118 | 82 | 36 |
| 1 | | Red | pinched | | | | | | | |
| Rep | 30 | Mirage ™ | Non-pre- | 10 | Nov. 21, 2019 | Jul. 24, 2019 | Aug. 29, 2019 | 120 | 84 | 36 |
| 1 | | Red | pinched | | | | | | | |
| Rep | 30 | Mirage ™ | Non-pre- | 11 | Nov. 25, 2019 | Jul. 24, 2019 | Aug. 29, 2019 | 124 | 88 | 36 |
| 1 | | Red | pinched | | | | | | | |
| Rep | 30 | Mirage ™ | Non-pre- | 12 | Nov. 21, 2019 | Jul. 24, 2019 | Aug. 29, 2019 | 120 | 84 | 36 |
| 1 | | Red | pinched | | | | | | | |
| Rep | 30 | Mirage ™ | Pre-Pinched | 1 | Nov. 19, 2019 | Jul. 24, 2019 | Aug. 29, 2019 | 118 | 82 | 36 |
| 1 | | Red | | | | | | | | |
| Rep | 30 | Mirage ™ | Pre-Pinched | 2 | Nov. 25, 2019 | Jul. 24, 2019 | Aug. 29, 2019 | 124 | 88 | 36 |
| 1 | | Red | | | | | | | | |
| Rep | 30 | Mirage ™ | Pre-Pinched | 3 | Nov. 21, 2019 | Jul. 24, 2019 | Aug. 29, 2019 | 120 | 84 | 36 |
| 1 | | Red | | | | | | | | |
| Rep | 30 | Mirage ™ | Pre-Pinched | 4 | Nov. 19, 2019 | Jul. 24, 2019 | Aug. 29, 2019 | 118 | 82 | 36 |
| 1 | | Red | | | | | | | | |
| Rep | 30 | Mirage ™ | Pre-Pinched | 5 | Nov. 19, 2019 | Jul. 24, 2019 | Aug. 29, 2019 | 118 | 82 | 36 |
| 1 | | Red | | | | | | | | |
| Rep | 30 | Mirage ™ | Pre-Pinched | 6 | Nov. 25, 2019 | Jul. 24, 2019 | Aug. 29, 2019 | 124 | 88 | 36 |
| 1 | | Red | | | | | | | | |
| Rep | 30 | Mirage ™ | Pre-Pinched | 7 | Nov. 21, 2019 | Jul. 24, 2019 | Aug. 29, 2019 | 120 | 84 | 36 |
| 1 | | Red | | | | | | | | |
| Rep | 30 | Mirage ™ | Pre-Pinched | 8 | Nov. 19, 2019 | Jul. 24, 2019 | Aug. 29, 2019 | 118 | 82 | 36 |
| 1 | | Red | | | | | | | | |
| Rep | 30 | Mirage ™ | Pre-Pinched | 9 | Nov. 25, 2019 | Jul. 24, 2019 | Aug. 29, 2019 | 124 | 88 | 36 |
| 1 | | Red | | | | | | | | |
| Rep | 30 | Mirage ™ | Pre-Pinched | 10 | Nov. 25, 2019 | Jul. 24, 2019 | Aug. 29, 2019 | 124 | 88 | 36 |
| 1 | | Red | | | | | | | | |
| Rep | 30 | Mirage ™ | Pre-Pinched | 12 | Nov. 21, 2019 | Jul. 24, 2019 | Aug. 29, 2019 | 120 | 84 | 36 |
| 1 | | Red | | | | | | | | |
| Rep | 32 | Mirage ™ | Non-pre- | 1 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 2 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 3 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 4 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 5 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 6 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 7 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 8 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 9 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 10 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 11 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 12 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 13 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 14 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 15 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 16 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 17 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 18 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 19 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 20 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 21 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 22 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 23 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 24 | Nov. 25, 2019 | Aug. 7, 2019 | Sep. 17, 2019 | 110 | 69 | 41 |
| 2 | | Red | pinched | | | | | | | |
| Rep | 32 | MirageT | Non-pre- | 25 | Dec. 2, 2019 | Aug. 7, 2019 | Sep. 17, 2019 | 117 | 76 | 41 |
| 2 | | Red | pinched | | | | | | | |
| Rep | 32 | Mirage ™ | Non-pre- | 26 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 27 | Dec. 2, 2019 | Aug. 7, 2019 | Sep. 17, 2019 | 117 | 76 | 41 |
| 2 | | Red | pinched | | | | | | | |
| Rep | 32 | Mirage ™ | Non-pre- | 28 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 29 | Dec. 2, 2019 | Aug. 7, 2019 | Sep. 17, 2019 | 117 | 76 | 41 |
| 2 | | Red | pinched | | | | | | | |
| Rep | 32 | Mirage ™ | Non-pre- | 30 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 31 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 32 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 33 | Dec. 2, 2019 | Aug. 7, 2019 | Sep. 17, 2019 | 117 | 76 | 41 |
| 2 | | Red | pinched | | | | | | | |
| Rep | 32 | Mirage ™ | Non-pre- | 34 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 35 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 36 | Nov. 21, 2019 | Aug. 7, 2019 | Sep. 17, 2019 | 106 | 65 | 41 |
| 2 | | Red | pinched | | | | | | | |
| Rep | 32 | Mirage ™ | Non-pre- | 37 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 38 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 39 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Non-pre- | 40 | Nov. 25, 2019 | Aug. 7, 2019 | Sep. 17, 2019 | 110 | 69 | 41 |
| 2 | | Red | pinched | | | | | | | |
| Rep | 32 | Mirage ™ | Pre-Pinched | 1 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 2 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 3 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 4 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 5 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 6 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 7 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 8 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 10 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 11 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 12 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 13 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 14 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 15 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 16 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 17 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 18 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 19 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 20 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 21 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 22 | Nov. 19, 2019 | Aug. 7, 2019 | Sep. 17, 2019 | 104 | 63 | 41 |
| 2 | | Red | | | | | | | | |
| Rep | 32 | Mirage ™ | Pre-Pinched | 23 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 24 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 25 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 26 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 27 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 28 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 29 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 30 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 31 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 32 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 33 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 34 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 35 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 36 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 37 | Nov. 21, 2019 | Aug. 7, 2019 | Sep. 17, 2019 | 106 | 65 | 41 |
| 2 | | Red | | | | | | | | |
| Rep | 32 | Mirage ™ | Pre-Pinched | 38 | Nov. 25, 2019 | Aug. 7, 2019 | Sep. 17, 2019 | 110 | 69 | 41 |
| 2 | | Red | | | | | | | | |
| Rep | 32 | Mirage ™ | Pre-Pinched | 39 | Dec. 2, 2019 | Aug. 7, 2019 | Sep. 17, 2019 | 117 | 76 | 41 |
| 2 | | Red | | | | | | | | |
| Rep | 32 | Mirage ™ | Pre-Pinched | 40 | Nov. 21, 2019 | Aug. 7, 2019 | Sep. 17, 2019 | 106 | 65 | 41 |
| 2 | | Red | | | | | | | | |

TABLE 3
Days to Finish: From Cutting Stick to Finish

| Variable | N | R² | Adj R² | CV |
|---|---|---|---|---|
| DTF Stick-Finish | 101 | 0.76 | 0.76 | 2.64 |

Analysis of variance table (Partial SS)

| | | | | | | | Week | Treat- | Vari- | | | | Mini- | Maxi- | wk to |
|---|---|---|---|---|---|---|---|---|---|---|---|---|---|---|---|
| S.V. | SS | df | MS | F | p-value | | Stuck | ment | able | n | Mean | S.D. | mum | mum | finish |
| Model | 2710.94 | 3 | 903.65 | 105.03 | <0.0001 | | 30 | Non-pre- | DTF | 11 | 120.9 | 2.59 | 118 | 124 | 17.5 |
| | | | | | | | | pinched | Stick- | | | | | | |
| | | | | | | | | | Finish | | | | | | |
| Week | 2630.05 | 1 | 2630.05 | 305.7 | <0.0001 | *** | 30 | Pre- | DTF | 11 | 120.7 | 2.72 | 118 | 124 | 17 |
| Stuck | | | | | | | | Pinched | Stick- | | | | | | |
| | | | | | | | | | Finish | | | | | | |
| Treatment | 22.2 | 1 | 22.2 | 2.58 | 0.112 | ns | 32 | Non-pre- | DTF | 40 | 109.5 | 3.11 | 106 | 117 | 15.5 |
| | | | | | | | | pinched | Stick- | | | | | | |
| | | | | | | | | | Finish | | | | | | |
| Week | 15.66 | 1 | 15.66 | 1.82 | 0.18 | ns | 32 | Pre- | DTF | 39 | 107.4 | 2.88 | 104 | 117 | 15.5 |
| Stuck * | | | | | | | | Pinched | Stick- | | | | | | |
| Treatment | | | | | | | | | Finish | | | | | | |
| Error | 834.53 | 97 | 8.6 | | | | | | | | | | | | |
| Total | 3545.47 | 100 | | | | | | | | | | | | | |

| Week Stuck | Means | n | S.E. | | |
|---|---|---|---|---|---|
| 32 | 108.47 | 79 | 0.3 | A | |
| 30 | 120.82 | 22 | 0.7 | | B |

Means with a common letter are not significantly different (p > 0.05)

TABLE 4
Days to Finish: From Pinch to Finish

| Variable | N | R² | Adj R² | CV |
|---|---|---|---|---|
| DTF pinch-finish | 101 | 0.86 | 0.86 | 4.12 |

Analysis of variance table (Partial SS)

| | | | | | | | Week | Treat- | Vari- | n | Mean | S.D. | Mini- | Maxi- | weeks to |
|---|---|---|---|---|---|---|---|---|---|---|---|---|---|---|---|
| S.V. | SS | df | MS | F | p-value | | Stuck | ment | able | | | | mum | mum | finish |
| Model | 5266.29 | 3 | 1755.43 | 204.04 | <0.0001 | | 30 | Non-pre- | DTF | 11 | 84.91 | 2.59 | 82 | 88 | 12 |
| | | | | | | | | pinched | pinch- | | | | | | |
| | | | | | | | | | finish | | | | | | |
| Week | 5187.59 | 1 | 5187.59 | 602.97 | <0.0001 | *** | 30 | Pre- | DTF | 11 | 84.73 | 2.72 | 82 | 88 | 12 |
| Stuck | | | | | | | | Pinched | pinch- | | | | | | |
| | | | | | | | | | finish | | | | | | |
| Treat- | 22.2 | 1 | 22.2 | 2.58 | 0.112 | ns | 32 | Non-pre- | DTF | 40 | 68.5 | 3.11 | 65 | 76 | 10 |
| ment | | | | | | | | pinched | pinch- | | | | | | |
| | | | | | | | | | finish | | | | | | |
| Week | 15.66 | 1 | 15.66 | 1.82 | 0.18 | ns | 32 | Pre- | DTF | 39 | 66.41 | 2.88 | 63 | 76 | 9.5 |
| Stuck * | | | | | | | | Pinched | pinch- | | | | | | |
| Treat- | | | | | | | | | finish | | | | | | |
| ment | | | | | | | | | | | | | | | |
| Error | 834.53 | 97 | 8.6 | | | | | | | | | | | | |
| Total | 6100.81 | 100 | | | | | | | | | | | | | |

| Week Stuck | Means | n | S.E. | | |
|---|---|---|---|---|---|
| 32 | 67.47 | 79 | 0.3 | A | |
| 30 | 84.82 | 22 | 0.7 | | B |

Means with a common letter are not significantly different (p > 0.05)

TABLE 5
Number of Branches Statistics
Number of Branches

| Variable | N | R² | Adj R² | CV |
|---|---|---|---|---|
| Number of Branches | 20 | 0.56 | 0.48 | 20.31 |

Analysis of variance table (Partial SS)

| | | | | | | |
|---|---|---|---|---|---|---|
| S.V. | SS | df | MS | F | p-value | |
| Model | 37.35 | 3 | 12.45 | 6.82 | 0.0036 | |
| Week Stuck | 0.45 | 1 | 0.45 | 0.25 | 0.6262 | ns |
| Treatment | 36.45 | 1 | 36.45 | 19.97 | 0.0004 | *** |
| Week Stuck*Treatment | 0.45 | 1 | 0.45 | 0.25 | 0.6262 | ns |
| Error | 29.2 | 16 | 1.83 | | | |
| Total | 66.55 | 19 | | | | |

| Week Stuck | Treatment | Variable | n | Mean | S.D. | Minimum | Maximum |
|---|---|---|---|---|---|---|---|
| 30 | Non-pre-pinched | Number of | 5 | 8 | 1.22 | 7 | 10 |
| | | Branches | | | | | |
| 30 | Pre-Pinched | Number of | 5 | 5.6 | 0.89 | 4 | 6 |
| | | Branches | | | | | |
| 32 | Non-pre-pinched | Number of | 5 | 8 | 1.22 | 7 | 10 |
| | | Branches | | | | | |
| 32 | Pre-Pinched | Number of | 5 | 5 | 1.87 | 2 | 7 |
| | | Branches | | | | | |
| Treatment | Means | n | S.E. | | | | |
| Pre-Pinched | 5.3 | 10 | 0.42 | A | | | |
| Non-pre-pinched | 8 | 10 | 0.42 | | B | | |

| | | | | | |
|---|---|---|---|---|---|
| Treatment | Means | n | S.E. | | |
| Pre-Pinched | 5.3 | 10 | 0.42 | A | |
| Non-pre-pinched | 8 | 10 | 0.42 | | B |

TABLE 6
Height Statistics
Height

| Variable | N | R² | Adj R² | CV |
|---|---|---|---|---|
| Height (cm) | 20 | 0.6 | 0.53 | 8.38 |

Analysis of variance table (Partial SS)

| S.V. | SS | df | MS | F | p-value | | |
|---|---|---|---|---|---|---|---|
| Model | 118.55 | 3 | 39.52 | 8.11 | 0.0017 | | |
| Week Stuck | 31.25 | 1 | 31.25 | 6.41 | 0.0222 | * | |
| Treatment | 76.05 | 1 | 76.05 | 15.6 | 0.0011 | ** | |
| Week Stuck*Treatment | 11.25 | 1 | 11.25 | 2.31 | 0.1482 | ns | |
| Error | 78 | 16 | 4.88 | | | | |
| Total | 196.55 | 19 | | | | | |
| Week Stuck | Treatment | Variable | n | Mean | S.D. | Minimum | Maximum |
| 30 | Non-pre- | Height | 5 | 26.4 | 1.82 | 24 | 29 |
| | pinched | (cm) | | | | | |
| 30 | Pre-Pinched | Height | 5 | 28.8 | 1.48 | 27 | 31 |
| | | (cm) | | | | | |
| 32 | Non-pre- | Height | 5 | 22.4 | 2.97 | 19 | 27 |
| | pinched | (cm) | | | | | |
| 32 | Pre-Pinched | Height | 5 | 27.8 | 2.28 | 25 | 31 |
| | | (cm) | | | | | |

Test: Tukey Alpha: = 0.05 LSD: = 2.16192
Error: 5.2500 df: 17

| Week Stuck | Means | n | S.E. | | |
|---|---|---|---|---|---|
| 32 | 25.1 | 10 | 0.72 | A | |
| 30 | 27.6 | 10 | 0.72 | | B |
| Means with a common letter are not significantly | | | | | |
| different (p > 0.05) | | | | | |

Test: Tukey Alpha: = 0.05 LSD: = 2.16192
Error: 5.2500 df: 17

| Treatment | Means | n | S.E. | | |
|---|---|---|---|---|---|
| Non-pre-pinched | 24.4 | 10 | 0.72 | A | |
| Pre-Pinched | 28.3 | 10 | 0.72 | | B |
| Means with a common letter are not significantly | | | | | |
| different (p > 0.05) | | | | | |

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit is 1, 2, 3, 4, 5, 6, 7, 8, 9, or 10 cm taller stems than a poinsettia pinched after planting and rooting.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit has about 1, 2, 3, 4, 5, 6, 7, 8, 9, or 10 fewer branches than a poinsettia pinched after planting and rooting.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days faster to retail size than a poinsettia pinched after planting and rooting.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit is a crop time reduced 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit is time from sticking to cyanthia opening and is reduced 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or 36 days.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the poinsettia variety is selected from the group of Mirage™ Red, Titan® Red, and Gemma™ Red.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the final container is a 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, or 24 inch pot.

In some embodiments, a method of growing a poinsettia with improved growth habit from a callused, pinched cutting is provided comprising the steps of cutting a stem tip cutting, callusing the cutting, pinching the stem tip before planting and planting the cutting in the final container, wherein the poinsettia produced by callusing and pinching before planting has an improved growth habit, wherein the improved growth habit is measured comparing plants grown from callused cutting and pre-pinched callus cuttings grown under the same conditions,

In some embodiments, a poinsettia plant with improved growth habit is provided produced by a method of growing from a callused, pinched cutting comprising the steps of cutting a stem tip cutting, callusing the cutting, pinching the stem tip before planting and planting the cutting in the final container.

In some embodiments, a method of producing a callused, pre-pinched Poinsettia cutting is provided comprising the steps of cutting a stem tip cutting, callusing the cutting and pinching the stem tip before planting.

In some embodiments, a callused, pre-pinched Poinsettia cutting is provided produced by a method comprising the steps of cutting a stem tip cutting, callusing the stem tip cutting, and pinching the stem tip before planting.

In some embodiments, a callused, pre-pinched Poinsettia cutting is provided.

### EXAMPLES

### Example 1

A standard Poinsettia cutting is 4.0 to 5.0 cm long when the cutting is harvested from the stock plant and has approximately four leaf nodes on the stem. Length is measured from the base of the cut stem to the shoot apical meristem. To create a pre-pinched callused Poinsettia cutting, the first step is to harvest a cutting from the stock plant that is 2.0 to 2.5 cm longer than a standard cutting (total length 6.0 to 7.0 cm) with one to three additional leaf nodes for a total of five to seven nodes. After harvest, the next step is callusing the cutting. The cutting of appropriate length and node count is stuck into a pre-made dibbled hole about 1 cm in diameter in an appropriate pre-wetted rooting medium (such as disinfected sand, rockwool, or peat moss). Rooting hormone [indole-3-butyric acid (IBA) or naphthaleneacetic acid (NAA)] may be applied as a liquid or powder dip to the base of the stem before sticking to accelerate the rooting process and improve uniformity. The rooting medium is usually contained in planting trays consisting of individual cells and these trays are on a raised bench in a greenhouse.

After sticking the cutting in the rooting medium, the cutting is misted with water regularly to retain turgidity. The ideal misting frequency should continually provide a film of water on the leaves and maintain humidity around 100%. Shade is applied over the propagation area to reduce stress on the cuttings and maintain 1,000-2,000 foot candles (200-300 μmol·m⁻²·s⁻¹) during the brightest part of the day. The 24-hour average daily temperature of the rooting medium should be 22-26° C. (72-78° F.). Optional fertilizers, fungicides, insecticides, and plant growth regulators can be applied.

Callus formation at the base of the cut stem begins about 7 to 10 days after sticking in the medium and root tips are visible 10 to 14 days after sticking, depending on media, air temperature, and medium temperature. Approximately 17 to 21 days after sticking, the cuttings have developed roots approximately 2 to 3 cm long, and are ready to be harvested, pinched, and packaged for shipping to the customer. In this process, a greenhouse worker removes the rooted callused cutting from the propagation medium and then rubs off any roots with their fingers, leaving only the callus at the base of the cutting. The next step is pinching the distal shoot apical meristem off the cutting, removing one to two leaf nodes and leaving a minimum of four leaf nodes on the cutting. The shoot-tip pinch can be done manually by hand or with a disinfected knife. The removed shoot-tip is discarded and the remaining basal portion of the cutting with a minimum of four leaf nodes is the finished product.

### Example 2

The objective of this experiment was to evaluate the effects of a novel pre-pinch callused cutting (CC) on the height, branch number, and days to finish of poinsettia (Euphorbia pulcherrima). Two product forms of poinsettia CC: Pre-pinched (PP) and Standard (STD) were tested in Gilroy, California on week 30 (Jul. 24 2019, replication 1) and week 32 (Aug. 7 2019, replication 2). The pre-pinched CC were callused and pinched before shipping to the testing site. The STD CC were only callused and not pre-pre-pinched.

Upon delivery of the cuttings, they were all direct stuck into 1-quart (950 mL volume) finished containers with 1 plant per pot. After rooting into the media, the STD CC were pinched 5 weeks after stick. The PP CC did not have to be pinched because cuttings were already pinched by the supplier, which saved 20% labor during finishing. The pinch date in table 2 is the date that the control cc plants where pinched in the experiment. No plant growth regulators were used for this experiment. Days to anthesis (open cyathia) were collected on 12 pots from replication 1 and 40 pots from replication 2. Plant height measurements and number of branches were taken on 5 plants from each product form within each replication.

All poinsettias were propagated under long-days to prevent flowering (13.5 hour day lengths with 4 hours of night interruption). The propagation greenhouse was maintained at a 23° C. day/21° C. night temperature and a daily light integral (DLI) of 11.8 mols/day. All plant material moved to short day, inductive day lengths (11.5 hrs.) week 35 (Aug. 27 2019) until anthesis. The short-day greenhouse temperatures were 23° C. day/20° C. night with a DLI of 8.6 mols/day.

The PP CC were rooted and ready to transfer to the short-day greenhouse for flower initiation 1 to 2 weeks earlier than the STD CC. However, in this experiment the PP CC remained under long-days until the STD CC could catchup and develop enough vegetative growth so both treatments could be transferred to short days at the same time. At the time of transfer, the PP CC were taller than STD CC. After the start of short days, all plants finished in 12 weeks. If the PP CC had been transferred sooner to short-days, plants would have flowered and shipped to market 1 to 2 weeks earlier, saving significant growing costs.

The PP CC were 4 cm taller at finish and had a mean of 3 less branches per plant than the standard treatment. Surprisingly, the PP CC produced acceptable retail quality plants even though they had on average 5.3 branches. Transferring the PP CC to short days 2 weeks earlier when they were ready would have likely reduced the 4 cm height difference between treatments. This experiment showed that the novel PP CC poinsettia can produce a marketable finished plant with 20% fewer labor inputs and up to 2 weeks shorter growing time.

TABLE 7
Replications 1 and 2 averages of plant height and number of
branches 18 weeks after sticking cuttings into 1-quart pots.

| | | |
|---|---|---|
| Treatment | Height (cm) | Number of Branches |

| | | | | |
|---|---|---|---|---|
| Pre-pinched CC | 28.3 | b* | 5.3 | a |
| Standard CC | 24.4 | a | 8.0 | b |

*Means within a column followed by the same letter are not significantly different according to Tukey comparison at P ≤ 0.05.

### Example 3

The objective of this experiment was to evaluate the effects of a novel pre-pinch callused cutting (CC) on the height, branch number, and days to finish of poinsettia varieties: Mirage™ Red, Titan® Red, and Gemma™ Red. A trial was performed in Monroeville, NJ, (Week 32-50, 2020) comparing standard callused cutting (CC) versus pre-pinched callused (PPCC) poinsettia cutting to evaluate its effects on flowering time and finish quality of Mirage™ Red, Titan® Red, and Gemma™ Red. Fifty plants were grown for each variety in each treatment. Average data values are shown in Table 8. The first shipment were direct stuck into 6.5″ pots and the second shipment were direct stuck into 4″ pots and grown inside a greenhouse at an Average Daily Temperature, ADT, of 72.05° F. (22.25° C.), Daily Light Integral, DLI, of 19 mols/day, and naturally decreasing day length of 14.5-10.5 hours. The 4″ and 6.5″ standard CC's were pinched to 5 and 7 nodes, respectively. Although the PPCC's had fewer branches, their bracts were more expanded, plants were taller, and the cyathia opened 1-2.5 weeks earlier than the standard CC; standard CC and PPCC received the same PGR regime.

These observations indicate that the PPCC's had a jump start in growing after the pinch versus the standard CC's, thus reaching finished shipping stage earlier. Overall, the trial contributed to further proving the technical concept of pre-pinching a callused poinsettia cutting to be able to produce a marketable plant in a shorter time period with good quality while reducing labor inputs

TABLE 8

| | | | | | | | | Crop |
|---|---|---|---|---|---|---|---|---|
| | | | | | | | | Time |
| | | | | | Average | Average | | (from |
| | Week | Pot | Variety | | Branch | Height | Flowering Date | stick |
| Expt. | Stuck | Size | Name | Treatment | Number | (cm) | (Open Cyathia) | date) |
| 3 | 32 | 6.5″ | Mirage ™ | Standard CC | 6 | 22.5 | Nov. 23, 2020 (wk 48) | 16 |
| | | | Red | | | | | |
| 3 | 33 | 6.5″ | Mirage ™ | Pre-Pinched | 4 | 26.5 | Nov. 19, 2020 (wk 47) | 14.4 |
| | | | Red | CC | | | | |
| 3 | 32 | 6.5″ | Titan ® Red | Standard CC | 6 | 19 | Nov. 17, 2020 (wk 47) | 15.1 |
| 3 | 33 | 6.5″ | Titan ® Red | Pre-Pinched | 4 | 26 | Nov. 13, 2020 (wk 46) | 13.6 |
| | | | | CC | | | | |
| 3 | 32 | 6.5″ | Gemma ™ | Standard CC | 6.5 | 33.5 | Dec. 9, 2020 (wk 50) | 18.3 |
| | | | Red | | | | | |
| 3 | 33 | 6.5″ | Gemma ™ | Pre-Pinched | 4 | 39 | Dec. 5, 2020 (wk 49) | 16.7 |
| | | | Red | CC | | | | |
| 3 | 34 | 4.0″ | Mirage ™ | Standard CC | 5 | 14 | Nov. 23, 2020 (wk 48) | 14 |
| | | | Red | | | | | |
| 3 | 35 | 4.0″ | Mirage ™ | Pre-Pinched | 4.5 | 20 | Nov. 19, 2020 (wk 47) | 12.4 |
| | | | Red | CC | | | | |
| 3 | 34 | 4.0″ | Titan ® Red | Standard CC | 5 | 15.5 | Nov. 17, 2020 (wk 47) | 13.1 |
| 3 | 35 | 4.0″ | Titan ® Red | Pre-Pinched | 4 | 17.5 | Nov. 13, 2020 (wk 46) | 11.6 |
| | | | | CC | | | | |
| 3 | 34 | 4.0″ | Gemma ™ | Standard CC | 5 | 21 | Dec. 14, 2020 (wk 51) | 17 |
| | | | Red | | | | | |
| 3 | 35 | 4.0″ | Gemma ™ | Pre-Pinched | 4 | 30 | Dec. 7, 2020 (wk 50) | 15 |
| | | | Red | CC | | | | |

What is claimed:

1. A method of growing a poinsettia with improved growth habit from a callused, pinched cutting comprising the steps of:
A) Cutting a stem tip cutting,
B) Callusing the cutting,
C) Pinching the stem tip before planting,
D) Planting the cutting in the final container,
wherein the poinsettia produced by callusing and pinching before planting has an improved growth habit.

2. The method of claim 1, wherein the improved growth habit is about 4 cm taller stems than a poinsettia pinched after planting and rooting.

3. The method of claim 1, wherein the improved growth habit has about 3 fewer branches than a poinsettia pinched after planting and rooting.

4. The method of claim 1, wherein the improved growth habit is 14 days faster to retail size than a poinsettia pinched after planting and rooting.

5. A plant produced from the method of claim 3.

6. A method of producing a callused, pre-pinched poinsettia cutting comprising the steps of cutting a stem tip cutting and callusing the cutting, pinching the stem tip before planting.

7. A callused, pre-pinched poinsettia cutting produced by the method of claim 6.
